(19) United States
(12) Patent Application Publication
(10) Pub. No.: US 2015/0232915 A1
(43) Pub. Date: Aug. 20, 2015

(54) Method for Real-Time Single Molecule Sequencing

(71) Applicant: Personal Genomics, Inc., Grand Cayman (KY)

(72) Inventors: Tsu-Ju Fu, Poway, CA (US); Shang-Chia Chang, Hsinchu County (TW); Jenn-Yeh Fann, Hsinchu County (TW)

(73) Assignee: Personal Genomics, Inc., Grand Cayman (KY)

(21) Appl. No.: 14/183,533

(22) Filed: Feb. 19, 2014

(51) Int. Cl. C12Q 1/68

(52) U.S. Cl. CPC C12Q 1/6806

(57) ABSTRACT

This invention is to describe a method for the determination of a nucleic acid sequence, in which it involves an enzyme, an enzyme complex or plural number of enzymes with more than one enzymatic activity and a set of nucleotide analogs, to achieve high signal readout accuracy in nucleic acid sequencing by making each signal to have a long signaling time span which allows a higher signal clarity.

### BACKGROUND OF THE INVENTION

Through continuous technological advancements, the detection of single molecules has become a reality. Several single-molecule measurement techniques, such as fluorescence correlation spectroscopy (FCS),^{1,2} direct observation using diffraction-limited optics,^{3,4} zero-mode waveguides (ZMW),⁵ and nanopore detection,⁶ have been developed for various applications. The methodology of single-molecule detection offers much higher sensitivity and provides more detailed information than its conventional bulk measurement counterparts. Optimized systems and methods for single-molecule detection have a great potential for accelerating the DNA sequencing technology. To achieve the single-molecule detection, an optical system can be applied to detect a very weak signal from a selectively excited molecule in a complicated environment. The fundamental elements to a successful detection in this approach rely on clear individual signals and well-defined inter-signal spacing as to generate a collective output of distinct signals.

Understanding the detailed mechanistic processes of DNA polymerization is a basic requirement to design a novel sequencing chemistry. A set of continuous steps are involved in DNA polymerization: First of all, the nucleotide substrates located in the vicinity of primer-template-polymerase complex randomly diffuse to the active site of DNA polymerase and carry out base-pairing trials to the template at the 3′-end of the primer inside the complex. In the following step, any nucleotide should achieve a base-pairing with the DNA template at the DNA polymerase active site, a pyrophosphate is cleaved off from the nucleotide substrate and a phospho-diester bond linkage is formed, catalyzed by DNA polymerase, between the 3′-end of the nascent DNA primer and the 5′-end of this substrate. Then, the last-joined nucleotide becomes the new 3′-end of the primer and the active site of polymerase moves forward to the next position of the template for a new repeating DNA synthesis cycle.

Up to today, the only source of commercially available instruments that perform optical real-time single molecule sequencing on the current market is Pacific Biosciences (PacBio; Palo Alto, Calif.). A comparison study has been recently published from the Wellcome Trust Sanger Institute in UK, which states that the PacBio's RS sequencer suffers greatly from its extremely high error rate (13%), which is the highest compared to the ones generated by those platforms of Ion Torrent's PGM (1.78%) and Illumina's Miseq (0.4%).⁷ As one carefully examines the current sequencing chemistry used by PacBio, each fluorescent dye is connected to the 5′-end of ribose through a polyphosphate linkage. Different lengths of polyphosphate have been tested and longer versions appear to be preferred for their better performances in the process of sequencing.^{8,9,10} The location of fluorescent labeling in the nucleotide, through a connection to the 5′-end polyphosphate of the nucleotide, is an unique approach adopted by PacBio's technology. As described in the proprietary zero-mode-waveguide (ZMW) design, only the fluorescent molecules in a defined volume can be activated and the DNA polymerase is purposely placed within this space.¹¹ In each compartment of the sequencing cells, it consists DNA polymerase, sequencing template, cognate primer, and all four base-specific fluorescent deoxynucleotide polyphosphates. Since the fluorescent molecules are diffusing in and out freely of the described volume in which the fluorophore activation light can reach, all the activated fluorescent signals of free moving labeled nucleotides within this volume are averaged out to a level of intensity that is considered as background. PacBio's technology has managed to place one DNA polymerase, at most, in each detection compartment. Every nucleotide substrate molecule in the vicinity of DNA-polymerase complex shares the same chance to interact with the complex. If any of those dye-carrying molecules is retained within this area by any reason, its fluorescent signal will become noticeable for detection. In theory, one would be able to detect the fluorescence signal that corresponds to a specific nucleotide substrate when it forms a base pair to the template at the active site of the DNA polymerase. Collectively, this is the basic idea of PacBio's detection technology.¹² Since the fluorophore is physically connected to the end of a polyphosphate group in each nucleotide substrate, the successfully reporting fluorescent signal that corresponds to an adequate base-pairing substrate begins at the moment when this substrate forms a base-pair with the template at the active site (bright phase) and the signal quickly dissipates after the moiety of dye and partial polyphosphate at the 5′-end of nucleotide substrate gets cleaved off from the DNA-polymerase complex and diffuses away. The subsequent event of a phosphodiester bond formation between the monophosphate nucleotide and the nascent primer strand is irrelevant to signal reporting (dark phase). By design, sequencing is achieved via a continuous recording of all incidents for each ephemeral fluorescence flash (bright phase). Since this detection method is very sensitive to the retention time of each fluorescent molecule in the “observing volume”, however, some of the unclear lingering of fluorescent molecules can produce false signals and consequently result in generating sequence insertion errors. From the empirical sequencing data collected by the described method, for instance, data shown in reference¹², it is important to note that not every peak in the time-resolved fluorescence intensity spectrum has the same intensity nor the time spans of all signals are identical. Furthermore, the spacing between every two consecutive peaks actually fluctuates quite a lot. A combination of these characteristics is, obviously, posing a serious challenger for any signal-reading algorithm. In real practices, besides the similar problems generated from the impurity of nucleotides, i.e., analog substrates lack fluorophore dyes, deletion errors in sequencing results can also be created by some of the dye-linked phosphates being cleaved off very quickly from their nucleoside moieties so that their fluorescent signals become too transient to be fully picked up. In addition, combining the frequent ambiguous tiny signals with the unpredictable spacing between peaks further worsen the chance of making correct base callings. In order to find an ultimate solution for such matters, fine-tuning the sequence recognition algorithms would not be as efficient as changing the fundamental sequencing chemistry.

### BRIEF SUMMARY OF THE INVENTION

This invention describes a method to produce distinct signals that allow more accurate readouts for single-molecule real-time DNA sequencing.

### DETAILED DESCRIPTION OF THE INVENTION

### 1. Methods of the Invention

### 1.1 Overview of the Methods

The rate of DNA replication can be as high as 1,000 bases per second by a DNA polymerase. With the current capability in single molecule detection, the replication speed is at least one to two orders of magnitude higher than what we can do for an accurate base-by-base recording of the DNA synthesis. In order to achieve a valid real-time single molecule sequencing, we have to slow down the rate of DNA replication. After examining the details of DNA synthesis processes, we have previously designed a sequencing system that utilizes the exonuclease activities of DNA polymerases to slow down the DNA synthesis proceeding (US Patent Application #20110300534). The described system involves a DNA template, a primer, a set of deoxynucleotide analogs with fluorescent tags at their 3′-ends, and a DNA polymerase that is capable of cleaving off the fluorophore moiety of the analog efficiently through its exonuclease activity. To describe the sequencing system in short: 1) one fluorescent nucleotide analog is added onto the nascent primer in the each step of DNA replication by a DNA polymerase, 2) the replication is stalled temperately due to the 3′-end fluorescent attachment, which could be an analog of a nucleotide, or a dinucleotide, 3) the identity of the added nucleotide is recorded by the fluorescent signal presented in its 3′-end attachment, 4) the 3′-end fluorescent moiety is resolved by the exonuclease activity of the DNA polymerase, and 5) 3′end hydroxyl group is restored and the last added nucleotide is ready for the next round of nucleotide addition. This “Pause & Go” sequencing design allows us not only to slow down the synthesis speed, but also to make the raw data easier to decipher by intensifying each signal strength (longer signaling time span) and, on some occasions, longer inter-signal spacing. Along the same trend of thought, we have noticed that another route can also be taken in the real time detection of a single molecule sequencing methodology by using the catalytic editing activities in some DNA polymerases or other equivalent enzymatic functions.

The catalytic editing activity of some DNA polymerases is one of the least studied enzymatic activities of DNA polymerases. A paper published by Canard et al. (1995)¹³ indicates that some of the DNA polymerases, i.e., Sequenase and HIV reverse transcriptase, are able to incorporate 3′-end esterified nucleotides (with various attachments) into the nascent primer and they can also perform an esterase-like activity (also called catalytic editing activity or 3′ intrinsic editing activity)¹⁴ to cleave off the 3′-end blocking attachments to resume DNA synthesis. From the same report, we have learned that the mentioned activity requires the presence of a correct nucleotide for the next position in primer-template-enzyme complex to carry out the described editing function. This observation results in a speculation that, as addressed in the reference¹³, the 3′-modified ends in nucleotides could not have been processed by an esterase activity before participating in DNA synthesis. Although additional experiments may be needed to further confirm the mechanistic details of such hydrolysis reaction, however, the published catalytic editing activity offers a good route to cleave off the 3′-end modification in a modified nucleotide. A corresponding material of Canard & Sarfati's work has been granted for a patent, which focuses on a step-wise analysis of hydrozylates in bulk for sequencing purpose.¹⁴ We consider the catalytic editing activity of some DNA polymerases can also be applied in the real-time single molecule sequencing, a method that has a unique usage very different from the relevant claims in Canard &Sarfati's prior patent.

### 1.2 Nucleotide Analogs

The choice of nucleotides used in real-time single-molecule sequencing has to ensure the replication will continue undisturbed during measurement. From the studies performed by PacBio, the selected DNA polymerase, i.e., phi 29 DNA polymerase, can utilize a nucleotide substrate with long polyphosphate (up to six phosphates) at the 5′-end.^{15,16,17} As for those using nucleotides with 3′-end modifications, there is very limited information available on the DNA polymerase's compatibility with these substrates. When one looks into the accumulated knowledge body on the replication terminators that are developed for the Sanger's Sequencing method, quite a few modifications have been made at the 3′-end in order to arrest the DNA amplification, permanent or reversible.^{18,19,20,21} Some of those nucleotide analogs that can fit in the “Pause & Go” sequencing methodology are listed as follows (Formula I and Formula II):

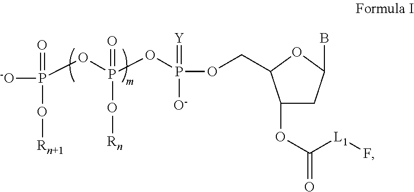

Wherein:
m and n are integers, m=n and 1≦m≦6;
Rₙ and Rₙ₊₁ (such as, R₁, R₂, . . . and R₇) represent the potential positions for a quencher attachment, the quenching moiety is added to prevent emission from the fluorescent dye attached to the 3′-end of the nucleotide, each of these position may or may not contain a linker L₂;
B is a base, which is chosen from adenine, cytosine, guanine, thymine, uracil, hypoxanthine, or 5-methylcytosine;
L₁ and L₂ are linkers, each of which can be alkyl, alkenyl, alkynyl, aryl, heteroaryl, heterocyclyl, polyethylene glycol, ester, amino, sulfonyl, or a combination of above functional groups;
Y is an oxygen (O) or a sulfur (S), a substitution of O in phosphate to S is to safeguard the nascent strand from a continuous 3′ to 5′ hydrolyzation by exonuclease; and
F is a photo-detectable label.

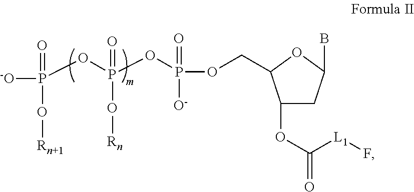

Wherein:
m and n are integers, m=n, and 1≦m≦6;
Rₙ and Rₙ₊₁ (such as, R₁, R₂, . . . and R₇) represent the potential positions for a quencher attachment, the quenching moiety is added to prevent emission from the fluorescent dye attached to the 3′-end of the nucleotide, each of these position may or may not consist a linker L₂;
B is a base, which is chosen from adenine, cytosine, guanine, thymine, uracil, hypoxanthine, or 5-methylcytosine;
L₁ and L₂ are linkers, each of which can be alkyl, alkenyl, alkynyl, aryl, heteroaryl, heterocyclyl, polyethylene glycol, ester, amino, sulfonyl, or a combination of the above functional groups;
Y is a methyl group (CH₃) or a boryl group (BH₂), a substitution of O in phosphate to CH₃ or BH₂ is to safeguard the nascent strand from a continuous 3′ to 5′ hydrolyzation by exonuclease; and
F is a photo-detectable label.

This group of nucleotide substrates will at least transiently arrest the reaction when they are used in DNA synthesis. Employing such nucleotide substrates in a single-molecule real-time sequencing system, i.e., PacBio's SMRT platform with minor modifications, each of the fluorescent signals will light up as soon as the substrate is base-pairing to the template at the active site of a DNA polymerase till the dye moiety is cleaved away from the DNA-polymerase complex by the catalytic editing activity of DNA polymerase. Once the 3′-end fluorophore-containing “blocking” group is cleaved off at the esterification site, a hydroxyl group is restored and the primer is ready for another round of nucleotide addition. In a similar approach, the dye-cleavage process may also be carried out by another enzyme or an enzyme moiety that is linked to a DNA polymerase. The sequencing process of the 3′-dye substrates involve at least two enzymatic activities, i.e., polymerization and catalytic editing activities, to complete each nucleotide addition cycle. Compared to the sequencing scheme using 5′-end dye-labeling (5′-dye) substrates, the 3′-end dye-labeling (3′-dye) nucleotide substrates that we described above offer a more accurate signal reporting due to their much more prolonged signals. Since the polymerization occurs at the 5′-end and the catalytic editing occurs at the 3′-end of the same nucleotide, it is still not clear if these two activities are actually correlated. Two interesting observations have been reported on this matter: 1) pre-incubation of 3′-esterified analogues with Sequenase in the absence of DNA primer/template doesn't make the enzyme pre-incubated analogues become replication-ready substrates for Taq polymerase in a regular primer extension reaction, 2) appearance of correct nucleotide for the next position in a stalled primer extension reaction is a prerequisite of the catalytic editing activity. These lines of evidence suggest that “the 3′ esterase and the polymerase active sites may be the same or at least be near one another and work closely together.”¹³ Using the molecular base-pairing event between a correct analogue and the template at the polymerase active site as a marking point, the fluorescent label of a 5′-dye analogue is cleaved off at the time very close to the marking point but the corresponding reaction of the 3′-dye analogue may not occurs until the next base-pairing takes place. In another word, the fluorescent signal in both cases begins at the moment when the substrate approaching the active site for base-pairing and this signal ends at the formation of phospodiester bond of the same round of base-pairing event for 5′-dye substrate, but it ends right before the next round of base-paring event for the 3′-dye substrate. The sequencing signal produced from the 3′-dye nucleotide is therefore longer and more distinguishable from the background and it allows the signal recognition algorithm weed out any false signals generated by the transient lingering of non-specific labeled molecules with less efforts. In this design, the fluorophore attachment at the 3′-end of the nucleotide not only serves as a signal reporter but also functions as a staller in polymerization. Only when the 3′-end blockage group is eventually removed by an enzymatic catalytic editing activity, the DNA synthesis resumes. Overall, the DNA synthesis is in a Pause-&-Go mode for every single nucleotide addition.

In a sequencing system, it may comprise a target nucleic acid, a primer nucleic acid comprising a sequence which is complementary to a region of the target nucleic acid, and a nucleic acid-polymerizing enzyme, and a set of photo-detectable nucleotide analogs, and a reaction buffer which contains acceptable salts. The most critical characteristic of this method is to use a set of base-specific photo-detectable probes, e.g., fluorescent labeling, attached to their 3′-ends that can be enzymatically cleaved from the nucleotides during replication. Using this design, the DNA polymerase, enzyme complex, or enzyme mix which comprises a 5′ to 3′ polymerization activity and a 3′-end catalytic editing activity are required to work with the 3′-end modified nucleotides to ensure the replication process is not interrupted to a stop. Each of those nucleotide analogs has a base moiety, which is capable of base-pairing with the template, and at least one photo-detectable label that can be correlated to the identity of the base that it carries. One important aspect of the analog is that the photo-label is linked to the 3′ position of the sugar through a coupler, which is an enzyme resolvable segment, and a length adjustable linker to connect the nucleotide and the labeling group so that this arrangement can allow the molecule to fit better into the activity center of a DNA polymerase. A main criterion of designing this line of analogs is that they must be able to serve as good substrates in DNA polymerization. On the other hand, the DNA polymerase may also need to be functionally optimized for an efficient utilization of those analogs by the protein engineering methodology, e.g., in vitro evolution (directed evolution). Different colors of (fluorescent) labeling are used in a base-specific fashion among the analogs. When one of these custom designed analogs is incorporated into the nascent strain of DNA during DNA polymerization (or primer extension), the photo-detectable label that is physically connected to the nucleotide can be activated, e.g., by UV radiation, and the analog identity which involves in the latest round of nucleotide addition is revealed. A further cleavage of the photo-label is carried out so that the nascent DNA strand is no longer blocked in replication at its 3′-end and the nascent DNA can be further extended. There have been some other methods presented previously, among which cleavable fluorophore labeling were used on either the 3′- or the 5′-end of their nucleotide analogs. For instance, Bruno Canard's group at Pasteur Institute (Paris, France),¹⁴ JingYue Ju's Lab at Columbia University (New York City, N.Y.),^{22,23} and Dongyun Shin & colleagues at Korea Institute of Science and Technology (Seoul, Korea),^{20,24} they have implemented stepwise stop-and-go procedures to decipher the identity of each nucleotide being added on the nascent strand DNA as a means of sequencing. However, the design of the presently described method allows one to make a continuous observation of the replication process in real time.

When feeding any substrate analog to an enzyme, there is always a concern of structural steric conflict between them. The molecules of interest here are nucleotide analogs that each has two major domains: a nucleotide and a fluorophore. In order to fit the analog better in the active site of a DNA polymerase, a linker between the two domains may play a critical role. A good example to illustrate this point is PacBio's efforts in selecting the proper nucleotide analogs used forits sequencing reactions, in which case different lengths of phosphate groups between the nucleotide and its fluorophore probe have been tested for an optimal performance in DNA synthesis.¹⁰ Other examples can also be found in various commercial fluorophore-labeled nucleotide analogs that are used in DNA labeling, examples such as those labeled nucleotide reagents offered by Life Technologies (Carlsbad, Calif.) and Glen Research (Sterling, Va.), different lengths of alkane, alkene, alkyne, or a combination of them are adopted in each compound to link the nucleotide with its fluorophore moiety. In light of the existing cases mentioned, linkers involved in the nucleotide analogs are useful tools to fine-tune the efficiency of their participation in DNA replications.

Other than linkers, the couplers that connect linker and nucleotide also have to be considered in the analog design. Since the labeling group attached at the 3′-end of the nucleotide analog will block the addition of the next nucleotide, it is critical to restore the extendable 3′-end between any two consecutive nucleotide additions. The sequencing method of interest here utilizes an enzymatic activity, or a combination of enzymatic activities, instead of involving non-continuous chemical treatments, to achieve a real-time observation of each nucleotide addition to the nascent strand. The couplers of choice have to be cleavable by an enzyme or a mixture of enzymes so that the sequencing process can be carried out continuously. By contrast, the method presented in the U.S. Pat. No. 5,798,210 in which a stepwise chemical means is illustrated to cleave the attached group. That is a method which may have a hard time to be integrated in a real time sequencing scheme. For our current purpose, there could be different choices for the functional group of coupler and the corresponding enzyme to digest the coupler that can result a leaving fluorophore and an polymerase-extendable DNA 3′-end. As for a practical reason, one can take the advantage of the catalytic editing activity of the DNA polymerase, as published by Canard, et al., to resolve the coupler-serving ester (—O—(CO)—) group to achieve the goal without the involvement of any additional enzymes. As for the couplers, judging from the data shown in the same paper, it appears that this catalytic editing activity works best on an ester linkage than other couplers such as amide and thiourea.¹³

### 1.2.1 Nucleotide Analog Details

As shown in Formula I & II, the base B may be, for example, a purine or a pyrimidine. For example, B may be an adenine, cytosine, guanine, thymine, uracil, or hypoxanthine. The base B may also be, for example, a naturally-occurring or synthetic derivative of a base, including pyrazolo(3,4-d)-pyrimidine; 5-methylcytosine (5-me-C); 5-hydroxymethyl cytosine; xanthine; hypoxanthine; 2-aminoadenine; 6-methyl or other alkyl derivative of adenine or guanine; 2-propyl or other alkyl derivative of adenine or guanine; 2-thiouracil; 2-thiothymine; 2-thiocytosine; 5-propynyl uracil; 5-propynyl cytosine; 6-azo uracil; 6-azo cytosine; 6-azo thymine; pseudouracil; 4-thiouracil; 8-halo (e.g., 8-bromo), 8-amino, 8-thiol, 8-thioalkyl, 8-hydroxyl and other 8-substituted adenine or guanine; 5-halo (e.g., 5-bromo), 5-trifluoromethyl and other 5-substituted uracil or cytosine; 7-methylguanine; 7-methyladenine; 8-azaguanine; 8-azaadenine; deazaguanine; 7-deazaguanine; 3-deazaguanine; deazaadenine; 7-deazaadenine; 3-deazaadenine; pyrazolo(3,4-d)pyrimidine; an imidazo(1,5-a)-1,3,5 triazinone; a 9-deazapurine; an imidazo(4,5-d)-pyrazine; a thiazolo(4,5-d)-pyrimidine; a pyrazin-2-one; a 1,2,4-triazine; a pyridazine; a 1,3,5 triazine; or the like.

In some embodiments, groups L₁ and L₂ are linkers. Suitable linkers include, for example, alkyl, alkenyl, alkynyl, aryl, heteroaryl, heterocyclyl, polyethylene glycol, ester, amino, sulfonyl linkers, or the like and the combinations of them. The linker may be any suitable linker which is nonreactive and which minimizes steric hindrance between the photo-detectable label and the remainder of the nucleotide analog.

Photo-detectable label F may be any moiety which can be attached to or associated with a nucleotide analog and which functions to provide a detectable signal. In some embodiments, the label is a fluorescent label, such as a small molecule fluorescent label. Useful fluorescent molecules (fluorophores) suitable as a fluorescent label include, but are not limited to: 1,5 IAEDANS; 1,8-ANS; 4-Methylumbelliferone; 5-carboxy-2,7-dichlorofluorescein; 5-Carboxyfluorescein (5-FAM); fluorescein amidite (FAM); 5-Carboxynapthofluorescein; tetrachloro-6-carboxyfluorescein (TET); hexachloro-6-carboxyfluorescein (HEX); 2,7-dimethoxy-4,5-dichloro-6-carboxyfluorescein (JOE); VIC®; NED™; tetramethylrhodamine (TMR); 5-Carboxytetramethylrhodamine (5-TAMRA); 5-HAT (Hydroxy Tryptamine); 5-Hydroxy Tryptamine (HAT); 5-ROX (carboxy-X-rhodamine); 6-Carboxyrhodamine 6G; 6-JOE; Light Cycler® red 610; Light Cycler® red 640; Light Cycler® red 670; Light Cycler® red 705; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; AFPs-AutoFluorescent Protein-(Quantum Biotechnologies); Texas Red; Texas Red-X conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamine-lsoThioCyanate); True Blue; TruRed; Ultralite; Uranin B; Uvitex SFC; WW 781; X-Rhodamine; XRITC; Xylene Orange; Y66F; Y66H; Y66W; YO-PRO-1; YO-PRO-3; YOYO-1; interchelating dyes such as YOYO-3, Sybr Green, Thiazole orange; members of the Alexa Fluor® dye series (from Molecular Probes/Invitrogen) which cover a broad spectrum and match the principal output wavelengths of common excitation sources such as Alexa Fluor 350, Alexa Fluor 405, 430, 488, 500, 514, 532, 546, 555, 568, 594, 610, 633, 635, 647, 660, 680, 700, and 750; members of the Cy Dye fluorophore series (GE Healthcare), also covering a wide spectrum such as Cy3, Cy3B, Cy3.5, Cy5, Cy5.5, Cy7; members of the Oyster® dye fluorophores (Denovo Biolabels) such as Oyster-500, -550, -556, 645, 650, 656; members of the DY-Labels series (Dyomics), for example, with maxima of absorption that range from 418 nm (DY-415) to 844 nm (DY-831) such as DY-415, -495, -505, -547, -548, -549, -550, -554, -555, -556, -560, -590, -610, -615, -630, -631, -632, -633, -634, -635, -636, -647, -648, -649, -650, -651, -652, -675, -676, -677, -680, -681, -682, -700, -701, -730, -731, -732, -734, -750, -751, -752, -776, -780, -781, -782, -831, -480XL, -481XL, -485XL, -510XL, -520XL, -521XL; members of the ATTO series of fluorescent labels (ATTO-TEC GmbH) such as ATTO 390, 425, 465, 488, 495, 520, 532, 550, 565, 590, 594, 610, 611X, 620, 633, 635, 637, 647, 647N, 655, 680, 700, 725, 740; members of the CAL Fluor® series or Quasar® series of dyes (Biosearch Technologies) such as CAL Fluor® Gold 540, CAL Fluor® Orange 560, Quasar® 570, CAL Fluor Red 590, CAL Fluor® Red 610, CAL Fluor® Red 635, Quasar® 570, and Quasar® 670.

In some embodiments, the photo-detectable label F interacts with a second photo-detectable moiety to modify the detectable signal provided by the first or second label, e.g., via Fluorescence resonance energy transfer (“FRET”; also known as Förster resonance energy transfer). In some embodiments, nucleotides incorporated into a nascent strand are detected using fluorescence resonance energy transfer (FRET)-based detection. For example, in some embodiments, a FRET-based method as described in U.S. Patent Application No. 2010/0035268 can be used. In such embodiments, a Quantum dot capable of acting as a fluorescence donor may be linked to a sequencing primer, and the nucleotide analogs used to synthesize the growing strand carry a label F which is a fluorescence acceptor. Incorporation of the fluorophore-labeled nucleotide analog into the growing nucleotide strand at a nucleic acid polymerizing enzyme active site is detected in real-time by detecting emission of the analog-linked fluorescence acceptor following fluorescence resonance energy transfer from the excited Quantum dot fluorescence donor. The identity of each incorporated nucleotide analog is determined by its fluorescent label, which is detectable while the analog is incorporated into the growing strand and until the attached moiety of the analog comprising the fluorescent label is removed by the catalytic editing activity of the polymerase.

In some embodiments, the nucleotide analog comprises a fluorescence quenching group Q. A fluorescence quenching group includes any moiety that is capable of absorbing the energy of an excited fluorescent label when located in close proximity to the fluorescent label and capable of dissipating that energy without the emission of visible light. Suitable fluorescence quenching groups include, for example, Deep Dark Quencher I (DDQ-I); 4-((4-(dimethylamino)phenyl)azo)benzoic acid, succinimidyl ester (DABCYL); Eclipse® dark quencher; Iowa Black® FQ; BHQ-1; QSY-7; BHQ-2; Deep Dark Quencher II (DDQ-II); Iowa Black® RQ; QSY-21; BHQ-3, and the like. A fluorescence quenching group Q may be linked to the gamma or beta phosphate of a nucleotide analog. A fluorescence quenching group may be connected via a linker L₂ to the gamma or beta phosphate of the nucleotide triphosphate analog, or any of the phosphate groups after the gamma position. Suitable linkers include, for example, alkyl, alkenyl, alkynyl, aryl, heteroaryl, heterocycloalkyl, polyethylene glycol, ester, amino, sulfonyl linkers, or the like and a combination of them. The linker may be any suitable linker which is nonreactive and which minimizes steric hindrance between the fluorescence quenching group and the remainder of the nucleotide analog.

The term “alkyl” as used herein refers to a saturated straight or branched hydrocarbon, such as a straight or branched group of 1-22, 1-8, or 1-6 carbon atoms, referred to herein as (C₁-C₂₂)alkyl, (C₁-C₈)alkyl, and (C₁-C₆)alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkenyl” as used herein refers to an unsaturated straight or branched hydrocarbon having at least one carbon-carbon double bond, such as a straight or branched group of 2-22, 2-8, or 2-6 carbon atoms, referred to herein as (C₂-C₂₂)alkenyl, (C₂-C₈)alkenyl, and (C₂-C₆)alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, etc.

The term “alkynyl” as used herein refers to an unsaturated straight or branched hydrocarbon having at least one carbon-carbon triple bond, such as a straight or branched group of 2-22, 2-8, or 2-6 carbon atoms, referred to herein as (C₂-C₂₂)alkynyl, (C₂-C₈)alkynyl, and (C₂-C₆)alkynyl, respectively. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl, etc.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this invention can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include, but are not limited to a monocyclic aromatic ring system, wherein the ring comprises 6 carbon atoms, referred to herein as “(C₆)aryl.”

The term “heteroaryl” as used herein refers to a mono-, bi-, or multi-cyclic, aromatic ring system containing one or more heteroatoms, for example one to three heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2 to 5 carbon atoms and 1 to 3 heteroatoms, referred to herein as “(C₂-C₅)heteroaryl.”

The term “heterocyclyl” or “heterocycle” as used herein refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The term “ester” refers to the structure —C(O)O—, —C(O)O—Rⱼ—, —RₖC(O)O—Rⱼ—, or —RₖC(O)O—, where O is not bound to hydrogen, and Rⱼ and Rₖ can independently be selected from alkoxy, aryloxy, alkyl, alkenyl, alkynyl, amide, amino, aryl, arylalkyl, cycloalkyl, ether, haloalkyl, heteroaryl, heterocyclyl. Rₖ can be a hydrogen, but Rⱼ cannot be hydrogen. The ester may be cyclic, for example the carbon atom and Rⱼ, the oxygen atom and Rₖ, or Rⱼ and Rₖ may be joined to form a 3- to 12-membered ring. Exemplary esters include, but are not limited to, alkyl esters wherein at least one of Rⱼ or Rₖ is alkyl, such as —O—C(O)-alkyl-, —C(O)—O-alkyl-, -alkyl-C(O)—O-alkyl-, etc. Exemplary esters also include aryl or heteoraryl esters, e.g. wherein at least one of Rⱼ or Rₖ is a heteroaryl group such as pyridine, pyridazine, pyrmidine and pyrazine, such as a nicotinate ester. Exemplary esters also include reverse esters having the structure —RₖC(O)O—, where the oxygen is bound to the parent molecular group. Exemplary reverse esters include succinate, D-argininate, L-argininate, L-lysinate and D-lysinate. Esters also include carboxylic acid anhydrides and acid halides.

The term “amino” as used herein refers to the form —NR_{d}Rₑ or —N(R_{d})Rₑ— where R_{d} and Rₑ are independently selected from alkyl, alkenyl, alkynyl, aryl, arylalkyl, carbamate, cycloalkyl, haloalkyl, heteroaryl, heterocyclyl, and hydrogen. The amino can be attached to the parent molecular group through the nitrogen. The amino also may be cyclic, for example, R_{d} and Rₑ may be joined together or with the N to form a 3- to 12-membered ring, e.g., morpholino or piperidinyl. The term amino also includes the corresponding quaternary ammonium salt of any amino group. Exemplary amino groups include alkyl amino groups, wherein at least one of R_{d} and Rₑ is an alkyl group.

The term “sulfonyl” as used herein refers to the structure RᵤSO₂—, where Rᵤ can be alkyl, alkenyl, alkynyl, aryl, cycloalkyl, and heterocyclyl, e.g., alkylsulfonyl. The term “alkylsulfonyl” as used herein refers to an alkyl group attached to a sulfonyl group. “Alkylsulfonyl” groups can optionally contain alkenyl or alkynyl groups.

“Alkyl,” “alkenyl,” “alkynyl,” and “amino” groups can be substituted with or interrupted by or branched with at least one group selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, thioketone, ureido, and nitrogen. The substituents may be branched to form a substituted or unsubstituted heterocycle or cycloalkyl.

As used herein, a “suitable substituent” refers to a group that does not nullify the synthetic or enzymatic utility of the compounds of the invention or the intermediates useful for preparing them. Examples of suitable substituents include, but are not limited to: C₁₋₂₂, C₁₋₈, and C₁₋₆ alkyl, alkenyl or alkynyl; C₁₋₆ aryl, C₂₋₅ heteroaryl; C₃₋₇ cycloalkyl; C₁₋₂₂, C₁₋₈, and C₁₋₆ alkoxy; C₆ aryloxy; —CN; —OH; oxo; halo, carboxy; amino, such as —NH(C₁₋₂₂, C₁₋₈, or C₁₋₆ alkyl), —N(C₁₋₂₂, C₁₋₈, and C₁₋₆ alkyl)₂, —NH((C₆)aryl), or —N((C₆)aryl)₂; formyl; ketones, such as —CO(C₁₋₂₂, C₁₋₈, and C₁₋₆ alkyl), —CO((C₆ aryl) esters, such as —CO₂(C₁₋₂₂, C₁₋₈, and C₁₋₆ alkyl) and —CO₂ (C₆ aryl). One of skill in art can readily choose a suitable substituent based on the stability and pharmacological and synthetic activity of the compound of the invention.

The term “acceptable salt(s)” refers to salts of acidic or basic groups that may be present in compounds used in the present compositions. Acceptable salts include salts which will not interfere with the reactions contemplated by the invention and are not otherwise undesirable. Acceptable salts do not differ in activity from their free base, and may include salts commonly referred to as pharmaceutically acceptable salts, which are non-toxic salts that retain the biological activity of the free base. Compounds included in the present compositions that are acidic in nature are capable of forming base salts with various cations. Examples of such salts include alkali metal or alkaline earth metal salts, including, for example, calcium, magnesium, sodium, lithium, and potassium salts. Acceptable salts may also include zinc, iron, ammonium, copper, manganese, aluminum salts and the like. Acceptable salts may also be those derived from organic non-toxic bases, and may include salts of primary, secondary, and tertiary amines, substituted amines, including naturally occurring substituted amines, cyclic amines and basic ion exchange resins, such as isopropylamine, tripropylamine, ethanolamine, 2-diethylaminoethanol, 2-dimethylaminoethanol, dicyclohexylamine, lysine, arginine, histidine, caffeine, procain, hydrabamine, choline, betaine, ethylenediamine, glucosamine, methylglucamine, theobromine, purines, piperazines, piperidine, polyamine resins and the like. In addition, salts may be formed from acid addition of certain organic and inorganic acids with basic centers of the purine, specifically guanine, or pyrimidine base. Finally it is to be understood that compounds of the present invention in their un-ionized as well as zwitterionic form and/or in the form of hydrates or solvates are also considered part of the present invention.

Combinations of a fluorophore and an interacting molecule or moiety, including quenching molecules or moieties, are known as “FRET pairs.” The mechanism of FRET-pair interaction requires that the absorption spectrum of one member of the pair overlaps the emission spectrum of the other member, the first fluorophore. If the interacting molecule or moiety is a quenching group, its absorption spectrum must overlap the emission spectrum of the fluorophore (Stryer, L., ANN. REV. BIOCHEM. 47: 819-846 (1978); C. R. Cantor and P. R. Schimmel, “Biophysical Chemistry—part II: Techniques for the Study of Biological Structure and Function,” W. H. Freeman and Co., San Francisco, U.S.A., 1980 (pages 448-455); and Selvin, P. R., METHODS IN ENZYMOLOGY, 246: 300-335 (1995)). Efficient FRET interaction requires that the absorption and emission spectra of the pair have a large degree of overlap. The efficiency of FRET interaction is linearly proportional to that overlap. (See Haugland, R. P., et al. PROC. NATL. ACAD. SCI. USA, 63: 24-30 (1969)). Typically, a large magnitude of signal (i.e., a high degree of overlap) is required. FRET pairs, including fluorophore-quenching group pairs, are therefore typically chosen on that basis.

Practical guidance is readily available in the literature for selecting appropriate FRET donor-acceptor pairs for particular probes, as exemplified by the following references: Pesce et al., Eds., “Fluorescence Spectroscopy,” Marcel Dekker, New York, 1971; White et al., “Fluorescence Analysis: A Practical Approach,” Marcel Dekker, New York, 1970. The literature also includes references providing exhaustive lists of fluorescent and chromogenic molecules and their relevant optical properties for choosing reporter-quencher pairs (see, for example, Berlman, HANDBOOK OF FLUORESCENCE SPECTRA OF AROMATIC MOLECULES, 2ND EDITION, Academic Press, New York, 1971; Griffiths, COLOUR AND CONSTITUTION OF ORGANIC MOLECULES, Academic Press, New York, 1976; Bishop, Ed., INDICATORS, Pergamon Press, Oxford, 1972; Haugland, HANDBOOK OF FLUORESCENT PROBES AND RESEARCH CHEMICALS, Molecular Probes, Eugene, 1992; Pringsheim, FLUORESCENCE AND PHOSPHORESCENCE, Interscience Publishers, New York, 1949. Further, the literature provides ample guidance for derivatizing reporter and quencher molecules for covalent attachment via common reactive groups that can be added to a nucleotide analog (see, e.g., Haugland (supra); U.S. Pat. Nos. 3,996,345 and 4,351,760).

An exemplified synthesis method of nucleotide with an ester linkage between the 3′-end of sugar and a fluorescent dye is available in the FIG. 2 of the U.S. Pat. No. 5,798,210.

### 1.3 Utilizing 3′ Modified Nucleotides by DNA Polymerases

Previously, some attempts were made to incorporate modified nucleotides containing bulky fluorescent dyes at the 3′-position with various DNA polymerases but none of them was satisfactory.^{25,26,27} It was speculated that the results might be due to the close proximity of the 3′-end position of deoxyribose and to the amino acid residues in the active sites of polymerases, as indicated in the simulation performed with the crystal structure data of T7 DNA polymerase.²⁷ Further efforts were extended to different DNA polymerases and various modified nucleotides of interest, however, some polymerases seemed to have the space in the active site pockets to accommodate certain sizes of attachments at the 3′-end of tested nucleotides. There had been at least two lines of data to support this approach, in each of which a nucleotide with a fluorescent dye attached to its 3′-end can be incorporated onto a primer by a DNA polymerase.^{13,20} Dr. Dongyun Shin, Dr. Dae-Ro Ahn and their colleagues published their work on utilizing Therminator II (New England Biolabs, Ipswich, Mass.) to incorporate 3′-labeled nucleotides in primer extension.^{28,20} Their corresponding patent application on this subject was also filed later.²⁴ Therminator™ II DNA Polymerase is a 9° N™ DNA Polymerase variant (D141A/E143A/A485L/Y409V). This enzyme is derived from its predecessor, Therminator DNA Polymerase, and differs by having one additional amino acid change (Y409V) that allows the enzyme more efficient incorporation of ribonucleotides²⁹ and nucleotides with modified 3′ functional groups.³⁰ It is noteworthy that Therminator II is capable of utilizing 3′-O-coumarin dTTPs, with an up to six-carbon linker between the ribose and its coumarin attachment, in the primer extension reactions.²⁰

Another piece of supporting data on this issue is published by Canard, Cardona, and Sarfati.¹³ This team has demonstrated that a group of 3′-modified nucleotides, including 3′-fluothioureido-dTTP, can be incorporated into DNA by DNA polymerases, such as Taq DNA polymerase, Sequenase 2.0 (United States Biochemicals, Cleveland, Ohio), and HIV-RT (Boehringer Mannheim). More intriguingly, as mentioned earlier, the latter two enzymes can hydrolyze the ester, and amido bonds at the nascent 3′ end of DNA to leave behind the hydroxyl and amine group, respectively. By comparison, Sequenase 2.0 can incorporate multiple 2′-deoxy-3′-anthranyloy-dNTPs consecutively to the end of a primer in an extension reaction but Taq DNA polymerase stops at only one nucleotide addition. Sequenase 2.0 is a genetically engineered mutant of T7 DNA polymerase by in vitro mutagenesis; it is different from its wild type by having virtually no 3′ to 5′ exonuclease activity. Therefore, this esterase-like catalytic editing activity of Sequenase 2.0 may not be relating to the 3′ to 5′ exonulease activity of T7 DNA polymerase. From the viewpoint of real time sequencing, the catalytic editing activities of some DNA polymerases can be of value to detach a base-specific fluorescent label at the 3′-end of each nucleotide, for example, through hydrolyzing ester bond, for a base reporting purpose when using fluoresce labeled nucleotides in sequencing. Every fluorescence-labeled nucleotide that involves in DNA primer extension, in a sequencing-by-synthesis approach, will present its fluorescent signal at the active site of DNA polymerase before the attached fluorescence label is cleaved off in the process of primer extension.

### EXAMPLES

A DNA molecule is sequenced according to the methods described herein. A solution of circular, single-stranded DNA molecules with an average length of 200 nt (nucleotides) at a concentration of 0.1 molecules per attoliter in a suitable sequencing reaction buffer is applied to a detection apparatus as described in U.S. Pat. Application entitled “Single-molecule Detection System and Methods,” and in U.S. Pat. App. No. 61/314,037, filed Mar. 15, 2010, from which the co-filed application entitled “Single-molecule Detection System and Methods” claims priority. Alternatively, the solution of circular, single-stranded DNA molecules is applied to a detection apparatus as described in U.S. application Ser. No. 12/801,503, filed Jun. 11, 2010; U.S. patent application Ser. No. 12/805,411, filed Jul. 29, 2010; U.S. Pat. Nos. 6,917,726; 7,170,050; and 7,486,865; and Eid, J., et al., SCIENCE, 323: 133-138 (2009).

The circular DNA molecules contain a known insert sequence of approximately 20 nucleotides 3′ to an unknown sample sequence. A sequencing primer complementary to the known insert sequence and four types of fluorescently labeled binucleotide analogs according to the invention are provided, wherein each of the four binucleotide analogs comprises a complementary base B₁ which is adenine, cytosine, guanine, or thymidine, respectively. In a plurality of detection sites in the detection apparatus, a ternary complex of a proofreading polymerase, DNA molecule, and sequencing primer is formed and the polymerase adds one fluorescently labeled nucleotide analog to the 3′ end of the sequencing primer.

In the plurality of detection sites, a fluorescently labeled nucleotide analog which associates with the active site of the polymerase and is incorporated into the growing (primer) strand is excited by excitation light from a light source coupled to the detection apparatus and emits fluorescent light. This fluorescent light is detected by the detection apparatus, which generates output signals to be processed to identify the base comprised by the binucleotide analog added to the sequencing primer. The fluorescent signal disappears when the polymerase cleaves the 3′-end attachment which comprises the fluorescent label from the growing strand, leaving the 3′-OH of the complementary group comprising base B.

The polymerase then adds another 3′-labeled nucleotide analog, which is detected as above. This cycle is repeated a sufficient number of times to acquire a sequencing read at least three times the length of the DNA molecule (i.e., the DNA molecule is sequenced and resequenced twice). The sequence of the DNA molecule is then obtained computationally by accepting or rejecting sequencing repeats and determining a consensus sequence from an alignment of the accepted repeats, as described in U.S. Pat. Pub. No. 2010/0121582, published May 13, 2010.

By using a catalytic editing activity of some DNA polymerases, a similar system is set up in this disclosure. A set of modified nucleotides, which has the general structure of Formula I or Formula II, is replacing the binucleotide analogs in the reaction described above to carry out the sequencing reactions. Instead of employing the 3′ to 5′ exonuclease activity, the catalytic editing activity of a DNA polymerase is capable of cleaving off the photo-labeled tag from the 3′-end of a nascent strand at a certain linkage point, e.g., ester group. This way, the 3′-end hydroxyl group of the growing strand is restored and the sequencing reaction can go on.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. The recitation of series of numbers with differing amounts of significant digits in the specification is not to be construed as implying that numbers with fewer significant digits given have the same precision as numbers with more significant digits given.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### Example 1

In this and the following examples, exemplary nucleotide analogs may comprise bases B, and/or groups Linker, Fluorophore, Y, R₁, R₂, . . . and Rₙ₊₁, each having an identity as described herein for nucleotide analogs having a structure of Formula I, supra.

A line of exemplary nucleotide triphosphate analogs have structure as shown in the following:

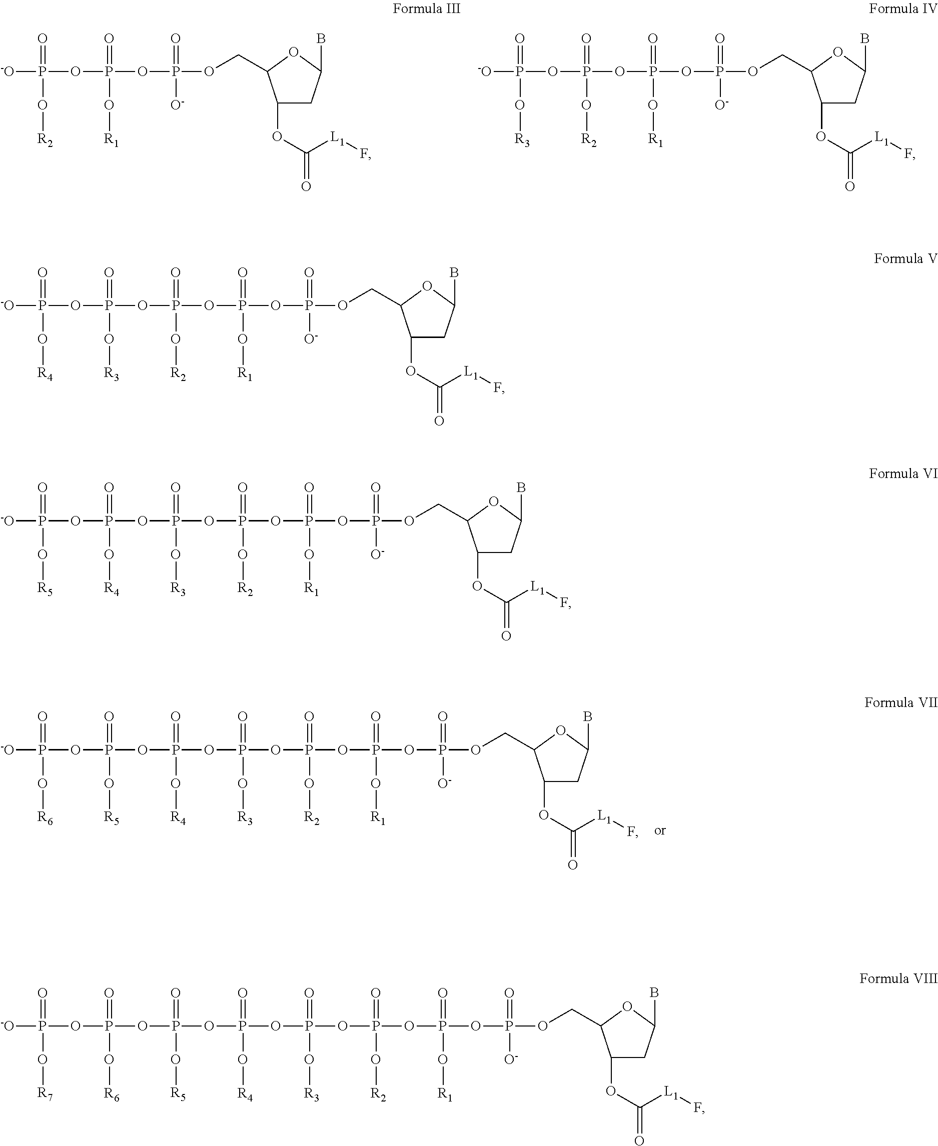

### Wherein:

R₁, R₂, R₃, R₄, R₅, R₆, and R₇ are each independently chosen from a hydrogen (H) or a fluorescence quencher, with or without a linker L₂;
F is a fluorescent dye;
B is a base, which is chosen from adenine, cytosine, guanine, thymine, uracil, hypoxanthine, or 5-methylcytosine;
L₁ and L₂ are linkers, which can be independently chosen from alkyl, alkenyl, alkynyl, aryl, heteroaryl, heterocyclyl, polyethylene glycol, ester, amino, sulfonyl, or a combination of some of groups mentioned above.

A schematic illustration of a single cycle of proofreading-dependent sequencing by synthesis using a binucleotide analog is provided in FIG. 1 of US patent Application No. 20110300534. A reaction complex comprising a proofreading polymerase, a target (template) strand, and a replicating strand are exposed to excitation light. An incoming binucleotide analog having adenine as the first base associates with the active site of the polymerase and base pairs with a thymine base in the target strand. The binucleotide analog is incorporated into the growing strand by the polymerase, whereupon the fluorescent label F is excited by the excitation light and emits a signal captured by a detector. As shown in the spectrogram, this signal remains detectable until the unpaired moiety of the analog (comprising the second base X which is not able to base pair with the subsequent base in the target strand and the fluorescent label F at its 3′-end) is cleaved from the growing strand by the exonuclease activity of the polymerase. The signal disappears as the labeled, unpaired moiety of the analog dissociates from the reaction complex. A similar case is elected as an example here. Instead of using the exonuclease activity of the DNA polymerase, an esterase activity of some DNA polymerases is involved in this example. A modified nucleotide has the structure of Formula III (with both R₁ and R₂ to be hydrogen) is replacing the binucleotide analog in the reaction described above. When an appropriate nucleotide analog is associated with the template and is incorporated into the growing strand by a polymerase, the fluorescent signal continues being detectable until the fluorophore gets cleaved off from the nascent strand by the enzyme at the ester linkage. The nucleotide added in this primer extension event can be identified by the kind of fluorescent probe attached on the modified nucleotide.

### Example 2

An exemplary triphosphate analog comprising a fluorescence quenching moiety Q has a structure of Formula IX:

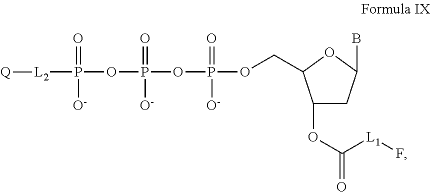

Wherein,
   Q is a fluorescence quenching moiety;
   B is a base, which is chosen from adenine, cytosine, guanine, thymine, uracil, hypoxanthine, or 5-methylcytosine;
   F is a fluorescent dye; and
   L₁ and L₂ are linkers, which can be alkyl, alkenyl, alkynyl, aryl, heteroaryl, heterocyclyl, polyethylene glycol, ester, amino, sulfonyl, or a combination of them.

An additional quencher (Q) is added to lower the general background due the appearances of those intact nucleotide-attached fluorescent dyes when they show up in the signal-detectable volume in the reaction space.

### Example 3

An exemplary triphosphate analog with a phosphorothioate in place of the alpha-phosphate of the triphosphate chain, thereby preventing processive 3′ to 5′ exonuclease activity of polymerase, has a structure as shown in Formula X:

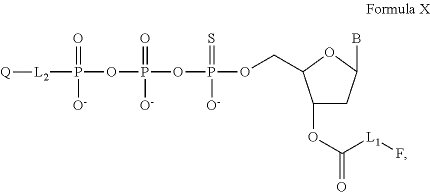

Wherein,
   Q is a fluorescence quenching moiety;
   B is a base, which is chosen from adenine, cytosine, guanine, thymine, uracil, hypoxanthine, or 5-methylcytosine;
   F is a fluorescent dye; and
   L₁ and L₂ are linkers, which can be alkyl, alkenyl, alkynyl, aryl, heteroaryl, heterocyclyl, polyethylene glycol, ester, amino, sulfonyl, or a combination of them.

A further consideration of some common DNA polymerases may also possess 3′ to 5′ exonuclease activities, the sulfur replacement at the alpha-phosphate of the modified nucleotide can be used to skew the reaction to less likely chew in (3′ to 5′) from the newly added nucleotide as to produce wrong sequencing outcomes.
¹Magde, D. et al, Phys. Rev. Lett. 29, 705 (1972).
²Mertz, J. et al., Optics Lett. 20,2532 (1995).
³Barak, L. S. & Webb, W. W. J. Cell Biol. 90, 595 (1981).
⁴Medina, M. A. & Schwille, P. Bioessays. 24, 758-64 (2002).
⁵Levene, M. J. et al., Science 299, 682 (2003).
⁶Venkatesan, B. M. & Bashir, R. Nat. Nanotechnol. 6, 615-24 (2011).
⁷Quail, M. et al., BMC Genomics. 13, 341 (2012).
⁸Y. Xu et al., U.S. Pat. No. 7,777,013 (2010).
⁹M. L. Metzker Nature Reviews Genetics 11, 31-46 (2010).
¹⁰Korlach, J. et al., Nucleosides, Nucleotides & Nucleic Acids 27, 1072-1082 (2008)
¹¹Korlach, J. et al., ProcNatlAcadSci USA. 105, 1176-81 (2008).
¹²Eid, J. et al., Science 323, 133-8 (2009).
¹³Canard, B. et al., ProcNatlAcadSci USA. 92, 10859-63 (1995).
¹⁴Canard, B. &Sarfati, S., U.S. Pat. No. 5,798,210.
¹⁵Korlach J. et. al. Nucleosides Nucleotides Nucleic Acids. 2008, 27, 1072-83.
¹⁶Y. Xu et al., U.S. Pat. No. 7,777,013 (2010).
¹⁷M. L. Metzker Nature Reviews Genetics 11, 31-46 (2010).
¹⁸Guo, J. et al., PNAS 105, 9145-50 (2008).
¹⁹Wu, J. et al., PNAS 104, 16462-7 (2007).
²⁰Kim, T.-S. et al., Chembiochem 11, 75-8 (2010).
²¹Metzker, M. L. et al., Nucleic Acids Res. 22, 4259-67 (1994).
²²Seo, T. S. et al., Proc. Natl. Acad. Sci. USA. 102, 5926-5931 (2005).
²³Ruparel, H. et al., Proc. Natl. Acad. Sci. USA. 102, 5932-7 (2005).
²⁴Shin, Dongyun et al., U.S. patent application Ser. No. 2011/0076679A1.
²⁵Turcatti, G. et al., Nucleic Acids Res. 36, e25 (2008).
²⁶Canard, B. & Sarfati, R. S. Gene 148, 1-6 (1994).
²⁷Welch, M. B. et al., Chem Eur. J. 5, 951-960 (1999).
²⁸Wu, J. et al., ProcNatlAcadSci USA. 104, 16462-7 (2007).
²⁹Gardner, A. F. & Jack, W. E. Nucleic Acids Res. 27, 2545-2555 (1999).
³⁰Ruparel, H. et al. Proc. Natl. Acad. Sci., USA, 26, 5932-5937 (2005).

What is claimed is:

1. A method of using nucleotide analogs to determine a nucleotide sequence of a target nucleic acid, comprising the steps of:
(a) providing a reaction complex comprising a target nucleic acid, a primer nucleic acid comprising a sequence which is complementary to a region of the target nucleic acid, and a nucleic acid-polymerizing enzyme, enzyme complex, or enzyme mix which comprises 5′ to 3′ polymerization activity and 3′ catalytic editing activity;
(b) contacting the reaction complex in a reaction buffer with nucleotide analogs, wherein each nucleotide analog comprises a base moiety and at least one label moiety comprising a photo-detectable label;
(c) the photo-detectable label is connected to a 3′ position of a sugar in the nucleotide analog by a couple group;
(d) allowing the nucleic acid-polymerizing enzyme, enzyme complex, or enzyme mix to incorporate one nucleotide analog to the primer nucleic acid to form a nascent primer strand via 5′ to 3′ polymerization activity of the nucleic acid-polymerizing enzyme, enzyme complex, or enzyme mix, whereby the label moiety and the base moiety of the nucleotide analog are incorporated in the nascent primer strand;
(e) detecting the photo-detectable label of the label moiety and determining an identity of the base moiety of the incorporated nucleotide analog;
(f) removing the label moiety of the incorporated nucleotide analog from the nascent primer strand via 3′ catalytic editing activity of the nucleic acid-polymerizing enzyme, enzyme complex, or enzyme mix; and
(g) repeating steps (d)-(f) to determine the nucleotide sequence of the target nucleic acid.

2. The method of claim 1, wherein the coupler group is an ester (—O—(CO)—).

3. The method of claim 2, wherein the photo-detectable label is a fluorescent dye and the label moiety includes an optional linker connecting the photo-detectable label to the coupler group —O—(CO)—, and the nucleotide analog has a structure as shown in Formula XI: 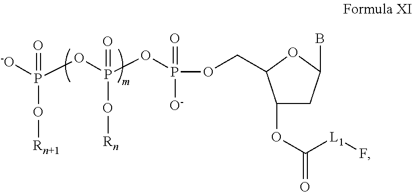
wherein
n=m, m and n are integers and 1≦m≦6;
Rₙ and Rₙ₊₁ (such as R₁, R₂, . . . and R₇) represent the potential positions for a quencher attachment, each is independently chosen from a hydrogen (H) or a fluorescence quencher, with or without linker L₂;
F is a fluorescent dye;
L₁ and L₂ are linkers, each independently is selected from alkyl, alkenyl, alkynyl, aryl, heteroaryl, heterocyclyl, polyethylene glycol, ester, amino, sulfonyl, or a combination thereof; and
B is chosen from adenine, cytosine, guanine, thymidine, uracil, hypoxanthine, or 5-methylcytosine.

4. The method of claim 3, wherein the nucleotide analog has a structure as shown in Formula I or Formula II: 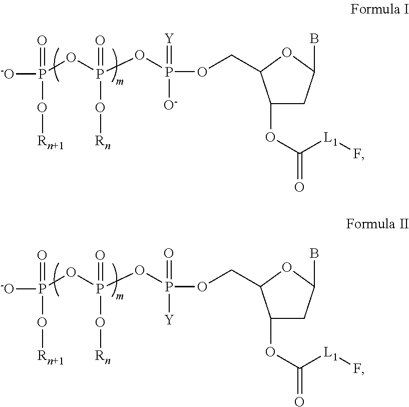
wherein
n=m, m and n are integers and 1≦m≦6;
Rₙ and Rₙ₊₁ (such as R₁, R₂, . . . and R₇) represent the potential positions for a quencher attachment, each is independently chosen from a hydrogen (H) or a fluorescence quencher, with or without linker L₂;
F is a fluorescent dye;
Y is an oxygen (O) or a sulfur (S);
L₁ and L₇ are linkers, each is independently selected from alkyl, alkenyl, alkynyl, aryl, heteroaryl, heterocyclyl, polyethylene glycol, ester, amino, sulfonyl, or a combination thereof; and
B is chosen from adenine, cytosine, guanine, thymidine, uracil, hypoxanthine, or 5-methylcytosine.

5. The method of claim 3, wherein the nucleotide analog has a structure as shown in Formula XII or Formula XIII: 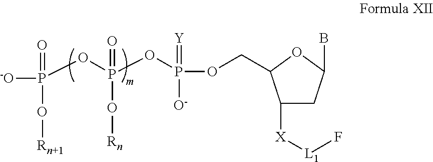
wherein,
n=m, m and n are integers and 1≦m≦6;
Rₙ and Rₙ₊₁ (such as R₁, R₂, . . . and R₇) represent the potential positions for a quencher attachment, each is independently chosen from a hydrogen (H) or a fluorescence quencher, with or without linker L₂;
F is a fluorescent dye;
Y is a sulfur (S) or an oxygen (O);
L₁ and L₂ are linkers, each independently is selected from alkyl, alkenyl, alkynyl, aryl, heteroaryl, heterocyclyl, polyethylene glycol, ester, amino, sulfonyl, or a combination thereof;
B is chosen from adenine, cytosine, guanine, thymidine, uracil, hypoxanthine, or 5-methylcytosine; and
X is a coupler group selected from —O—, —S—, —HN—, —PO₄⁻—, —COO—, —CO—, —NH(CS)NH—, or —NHCO—. 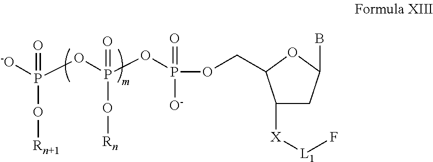
wherein,
n=m, m and n are integers and 1≦m≦6;
Rₙ and Rₙ₊₁ (such as R₁, R₂, . . . and R₇) represent the potential positions for a quencher attachment, each is independently chosen from a hydrogen (H) or a fluorescence quencher, with or without linker L₂;
F is a fluorescent dye;
Y is a methyl group (CH₃) or a boryl group (BH₂);
L₁ and L₂ are linkers, each is independently selected from alkyl, alkenyl, alkynyl, awl, heteroaryl, heterocyclyl, polyethylene glycol, ester, amino, sulfonyl, or a combination thereof;
B is chosen from adenine, cytosine, guanine, thymidine, uracil, hypoxanthine, or 5-methylcytosine; and
X is a coupler group selected from —O—, —S—, —HN—, —PO₄⁻—, —COO—, —CO—, —NH(CS)NH—, or —NHCO—.

6. The method of claim 1, wherein the photo-detectable label is a fluorophore.

7. The method of claim 3, wherein the nucleotide analog has 4-12 phosphate groups at the 5′-end of the nucleotide analog.

8. The method of claim 4, wherein the nucleotide analog has 4-12 phosphate groups at the 5′-end of the nucleotide analog.

9. The method of claim 5, wherein the nucleotide analog has 4-12 phosphate groups at the 5′-end of the nucleotide analog.

10. The method of claim 6, wherein the nucleotide analog has 4-12 phosphate groups at the 5′-end of the nucleotide analog.
